(19) United States
(12) Patent Application Publication
(10) Pub. No.: US 2021/0207843 A1
(43) Pub. Date: Jul. 8, 2021

(54) An Apparatus for Handling Air and System Thereof

(71) Applicant: AIRSMART (IAS) PTY LTD, Melbourne, Victoria (AU)

(72) Inventor: David Marko Filipovic, Docklands (AU)

(73) Assignee: AIRSMART (IAS) PTY LTD, Melbourne Victoria (AU)

(21) Appl. No.: 16/759,930

(22) PCT Filed: Nov. 2, 2018
(86) PCT No.: PCT/AU2018/000213

§ 371 Date: Apr. 28, 2020

(30) Foreign Application Priority Data
Nov. 3, 2017 (AU) 2017904477

(51) Int. Cl. F24F 13/20; F24F 8/158; F24F 8/167; F24F 8/108; F24F 8/22; F24F 13/28; F24F 11/46

(52) U.S. Cl. CPC F24F 13/20; F24F 8/158; F24F 8/167; F24F 11/46; F24F 8/22; F24F 13/28; F24F 8/108

(57) ABSTRACT

An apparatus for handling air in an enclosed space, the enclosure having a cover, an inlet formed in the cover and an outlet, the air being directable from the inlet to the outlet and, when the air is so directed, to change at least one characteristic of the air wherein the apparatus is adapted to receive purification means for purifying the air such that, when the purification means is so received, the air directed through the inlet is purified on entry into the enclosure.

### CROSS REFERENCE TO RELATED PATENT APPLICATION

The present application claims priority from Australian Provisional Patent Application No. 2017904477, filed on Nov. 3, 2017, the entire contents of which are incorporated herein by reference.

### FIELD OF THE DISCLOSURE

The present invention relates to an apparatus for handling and changing at least one characteristic of air in an enclosed space. The invention also relates to a system comprising the apparatus.

### BACKGROUND OF THE DISCLOSURE

Ducted systems for heating and cooling residential buildings have heating and cooling components which are typically concealed in confined spaces, such as the ceiling of the building. To maintain, repair or replace such components, these confined spaces can be difficult to access easily, can be at extreme temperatures and often have poor air quality.

Furthermore, previous ducted systems are unable to use high-quality filters which have the tendency to clog easily and have to be replaced on a regular basis. Moreover, the fans used to direct the air in these ducted systems are unable to cope with the demands of a high-quality filtration system which can necessitate frequent maintenance or repair. In addition, these fans are unable to adjust their output to maintain the required airflow as filters increase resistance as they capture pollutants. The reduced air flow which results from use of these fans also undesirably reduces the heating and cooling capacity and effectiveness of these ducted systems.

Examples of the inventions desirably avoid or at least ameliorate the problems described above.

### SUMMARY OF THE DISCLOSURE

According to a first aspect of the present invention, there is provided an apparatus for handling air in an enclosed space, the enclosure having a cover, an inlet formed in the cover and an outlet, the air being directable from the inlet to the outlet, and, when the air is so directed, to change at least one characteristic of the air, wherein the apparatus is adapted to receive purification means for purifying the air such that, when the purification means is so received, the air directed through the inlet is purified on entry into the enclosure.

According to the invention, the at least one characteristic of air includes one or more of the following: temperature, rate of air flow, humidity, air quality.

According to the invention, the cover is arranged to open to facilitate servicing.

According to the invention, wherein servicing includes maintenance, replacement or repair of the purification means. In a preferred embodiment, the servicing includes maintenance, replacement or repair of the apparatus including any fans, coils or electronics.

According to the invention, the inlet is defined as at least one aperture.

According to the invention, the purification means is positioned internal to the cover and adjacent to the inlet.

According to the invention, the apparatus is mountable within structure of a building.

In an embodiment, the apparatus is wall-mountable. Preferably, the apparatus is mountable in a cavity defined by walls of the enclosed space. More preferably, the apparatus is mountable at floor level. Alternatively, the apparatus is mountable in a ceiling or floor space.

According to the invention, the arrangement for opening the cover is a slidable arrangement such that the cover and slidable arrangement thereby define a drawer.

According to the invention, the apparatus is adapted to be accessible via an opening in the structure of the building wherein the slidable arrangement extends through the opening such that the cover is slidably movable from a retracted position where the cover occludes the opening and an extended position where the cover permits servicing of the apparatus.

Preferably, the structure of the building includes one or more walls, a ceiling or a floor.

According to the invention, the cover is defined as a panel. Preferably, the panel is a fascia having at least one opening.

According to the invention, the cover is securable in the retracted position.

According to the invention, changing the characteristic of air includes reduction of pollutant content.

According to the invention, the pollutant content includes particulates or volatile organic compounds. The pollutant content preferably includes smoke, diesel or petrol fumes, allergens, toxins and pathogens.

According to the invention, the filter includes one or more of a selection of: filter with a

Minimum Efficiency Reporting Value (MERV) level of at least 13, pre-filter, HEPA filter, carbon filter. Preferably, the purification means includes a filter with a MERV of between 13 to 16.

According to the invention, the apparatus has a fan configured to be operable at high air resistances for directing air from the inlet to the outlet and any purification means so received therebetween, and wherein the apparatus is configured to detect a pressure differential or resistance across the received purification means, and when the pressure differential is at a pre-determined level, the apparatus is configured to operate the fan at a higher rate thereby maintaining the flow of air.

According to the invention, the purification means utilises short-wavelength ultraviolet radiation (UVR) and/or photocatalytic oxidation technology (PCO). Preferably, the ultraviolet radiation is short-wavelength ultraviolet radiation (UVC).

According to the invention, the purification means is for purifying the breathable air.

According to the invention, changing the characteristic of air includes changing the CO, CO₂ or O₂ percentage content.

According to the invention, the apparatus includes a further inlet for allowing air external to the enclosed space or from the or a ceiling space to enter the enclosure.

According to the invention, the apparatus includes a purification means. Preferably, the purification means is positioned in the enclosure internal to the cover and adjacent to the inlet.

According to a second aspect of the present invention, there is provided a system for handling air and circulating that air about an enclosed space of a building, the system having the apparatus as described above, wherein the fan directs the air from the outlet via one or more ducts into the enclosed space into the inlet of the enclosure thereby circulating the air about the enclosed space.

According to the invention, the system further includes at least one sensor for detecting characteristics of the air.

According to the invention, the system further includes a controller coupled to the sensor(s) and the air characteristic changing means, wherein the controller provides signals to the changing means based on signals received from the sensor(s) or external signals associated with environmental monitoring to change the characteristic(s) of the air.

According to the invention, the sensor(s) is adapted to detect a reduction in efficiency of the purification means, and when the reduction is so detected, the sensor is adapted to provide a signal to the controller to send a further signal to service the purification means.

According to the invention, the further signal includes a signal that the purification means requires replacement.

### BRIEF DESCRIPTION OF THE DRAWINGS

The present invention will now be described, by way of non-limiting example only, with reference to the accompanying drawings, in which:

FIG. 1 is a front perspective view of an apparatus for changing at least one characteristic of air in an enclosed space, the apparatus having an enclosure with a cover according to an embodiment of the present invention;

FIGS. 2 and 3 are front and rear perspective views of the apparatus of FIG. 1 with the cover in an open position for facilitating servicing;

FIG. 4 is a rear perspective of the apparatus of Figures without part of the enclosure for ease of explanation;

FIG. 5 is an exploded view of the apparatus of FIG. 1;

FIG. 6 is an example plan view of a residential building with the system of FIG. 6 for circulating and changing at least one characteristic of air in an enclosed space; and

FIGS. 7 and 8 are example schematic views of a system for changing at least one characteristic of air in an enclosed space having the apparatus of FIGS. 1 to 5.

### DETAILED DESCRIPTION OF THE DRAWINGS

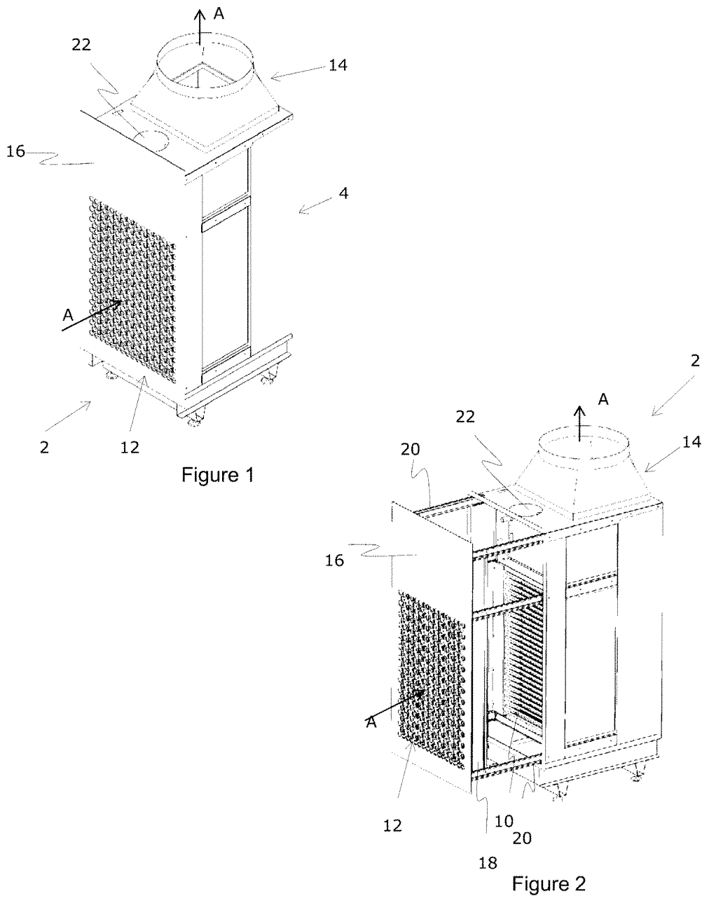

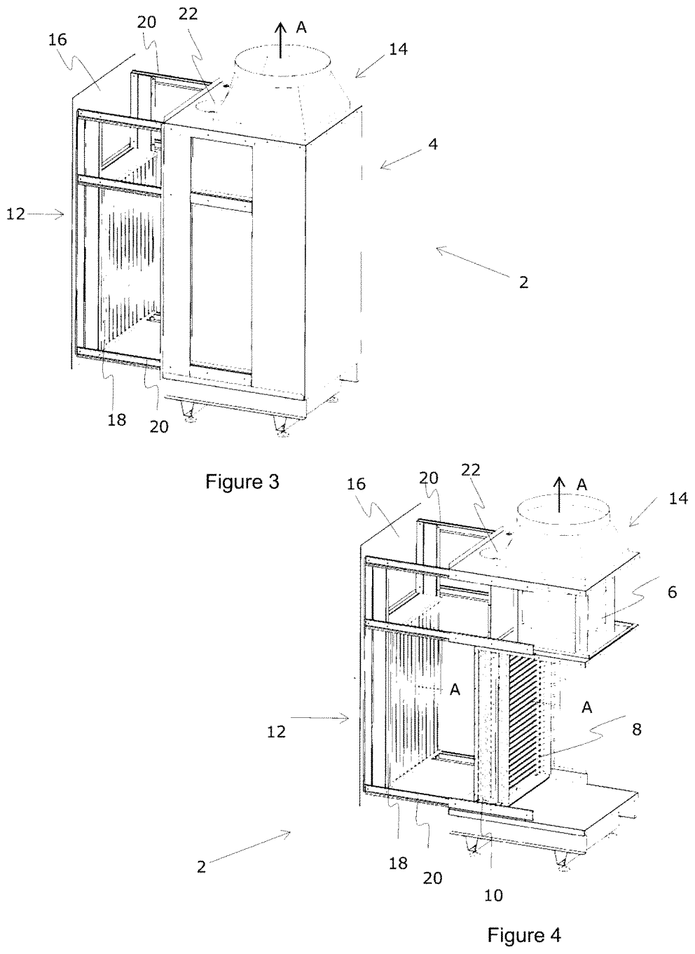

FIGS. 1 to 8 show various views of an apparatus 2 for handling and changing at least one characteristic of air according to an embodiment of the present invention, and in particular an apparatus 2 which is configured to facilitate servicing. The enclosure 4 has an inlet 12 and an outlet 14 and the apparatus is adapted to direct air through the enclosure 4 by a means for directing air 6, for example a fan. The direction of air flow is indicated by arrows ‘A’. The apparatus 2 is also adapted to change the at least one characteristic of the air by air characteristic changing means, for example a heating coil or a cooling coil, which will be discussed in more detail below.

The enclosure 4 has a cover 16 which is arranged to be openable from a closed position to an open position where the means for changing a characteristic of air and/or air directing means 6, 8, 10 contained within the enclosure 4 are accessible for facilitating servicing, i.e. for maintenance, replacement or repair. In the closed position, the cover 16 protects the air characteristic changing and/or directing means 6, 8, 10 and also hides them from view.

The characteristics of the air that can be changed include temperature i.e. heating and/or cooling, humidity and the quality of the air in particular, oxygen or carbon dioxide content, and pollutant content including pollutants such as particulates or volatile organic compounds. As such, the air characteristic changing means 8, 10, 18 can include heating or cooling coils and filters 18 or humidifier or dehumidifiers (not shown).

In the example of FIGS. 1 to 5, the cover 16 of the enclosure 4 is defined as a panel, which is slidably movable relative to the remainder of the enclosure 4, via multiple rail arrangements 20 connected to the enclosure 4 which extend transverse to the cover 16. As illustrated in FIGS. 1 to 5, the cover 16 in the open position is parallel to and spaced apart from the cover 16 when it is in the closed position. As such, the cover 16 and rail arrangement 20 form a drawer in which at least one purification means 18 can be located adjacent to the inner surface of the cover 16. Thus the quality of the air is improved and circulated through the enclosed space 1 constantly for the purpose of purifying the breathable air for the well-being of the occupants therein. In prior art heating and cooling systems, filters are typically used for protecting the heating coil rather than purifying the breathable air.

Advantageously, the purification means 18, for example filters, are conveniently accessible for ease of replacement, cleaning or repair simply by moving the cover 16 to the open or extended position, for example, by opening the drawer defined by the cover 16 and rail arrangements 20.

The fan 6 extracts air from the enclosed space 1 through the inlet 12 and directs it to the outlet 14. As exemplified in FIGS. 1 to 5, the inlet 12 can be in the form of at least one aperture in the cover 16 of the enclosure 4, and the air returning through the inlet 12 passes through the purification means 18 located adjacent to the inner surface of the cover 16, thereby purifying the air. The air expelled from the outlet 14 is directable via ducts 28 to various portions of the enclosed space 1. The apparatus 2 also has a second inlet 22 which allows outside air to be directed into the enclosure 4 via a duct, one end of the duct connected to the outside air inlet 22 and the other end of the duct being open external to the enclosed space 1. A further fan (not shown) can be provided to draw the outside air into the enclosure 4.

In another embodiment, the apparatus 2 can have a further inlet (not shown) which allows air in the ceiling space to be directed into the enclosure 4 via a further duct, one end of the duct being connected to the further inlet and the other end of the duct being open to the ceiling space. A second further fan can be provided to draw the air from the ceiling space into the enclosure 4. The air in the ceiling space is typically at a temperature which is different from the air external to the building and the air in the enclosed space 1. For example, the air in the ceiling space may be warmer than the external air and therefore by selecting to direct warmer air from the ceiling space in preference to cooler external air, heat can be recovered from the ceiling space air and obviates the need to re-heat the air at least partially before it is directed into the enclosed space 1. Similarly, the air in the ceiling space may be cooler than external air and therefore reduces the need to cool the ceiling space air at least partially before it is directed into the enclosed space 1. In a preferred embodiment, the apparatus 2 is configured to be able to select between directing air from the ceiling space or air from the external environment depending on which will allow efficient heat or cool recovery.

The quality of the air can be improved by using purification means such as filters that can remove particulates. As an example, one example of filters is that that can remove particles which are 2.5 microns or smaller, known as fine particles, and are breathable into the lungs, causing lung tissue damage. Examples of fine particles include smoke from cigarettes, fires or cooking, and diesel or petrol particulates. The controlling means can also have at least one filter which removes particles that are 2.5 microns or larger, known as coarse particles. Coarse particles do not penetrate the lungs as deeply as fine particles but many of them are allergens, toxins or pathogens, such as dust mite dust, pollens, carpet or clothing fibres, bacteria and mould spores.

Examples of filters usable in this apparatus to remove particulates includes mechanical air filters (mesh, screen, woven or filled material), or electrostatic filters or precipitators. Air passes through mechanical air filters to remove the particulates, which include filters such as activated carbon air filters, high efficient particulate air (HEPA) filters, filters having Zeolite or other such porous material. Electrostatic filters attract particles using an electrostatic charge and extracts them via a wire mesh.

The apparatus can also include filters, for example activated carbon air filters, to filter out gaseous pollutants such as volatile organic compounds, commonly known as VOCs, which accumulate in enclosed spaces from the evaporation of carbon based chemicals in our environment, such as paints, cleaning products, furniture and building materials. Examples of these pollutants include formaldehyde, nitrogen dioxide, and ozone.

The purification means can include, but is not limited to, a device which purifies by irradiation of ultraviolet radiation (UVR) and/or photocatalytic oxidation (PCO). When ultraviolet radiation is used to irradiate air, food products, liquids or surfaces, it can destroy harmful micro-organisms without chemicals or harmful side-effects. In a preferred embodiment, the ultraviolet radiation used may be short-wavelength ultraviolet irradiation (UVC) which is ultraviolet light with a wavelength of 200 to 400 nm. Photocatalytic oxidation is a method of purifying air by using broad-spectrum ultraviolet light which reacts with a thin film of titanium dioxide to create hydroxyl radicals and super-oxide ions that oxidise VOCs, toxic gases and chemicals into safer compounds such as carbon dioxide and water.

In a preferred example, the apparatus 4 has three filters 18, the filters being a pre-filter, a carbon filter and a filter with at least a Minimum Efficiency Reporting Value (MERV) 13 rating. A filter with a MERV rating of 13 is able to filter particulates with a size of 1 micron or greater, while a filter with a MERV rating of 16 or a HEPA filter is able to filter particulates with a size of 0.3 microns or greater. This three filter system advantageously provides a very high breathable air quality when air is handled by the apparatus 2. More preferably, the apparatus also includes three filters and a purification means utilising ultraviolet irradiation (UVR) and/or photocatalytic oxidation technology (PCO).

In a particular preferred embodiment, one of the afore-mentioned filters has a MERV rating of 16 or greater. As these filters having MERV ratings of between 13 and 16 or greater can screen such fine particulates, it can be easily clogged or blocked. Therefore, the fan 6 which directs air through said filter is configured to operate under high static air resistance thereby preventing the fan from requiring frequent maintenance or replacement. Further, the filter with a MERV rating of 13 or greater, can include at least one sensor which can detect if there is a pressure differential between one side of the filter to the other, which can indicate blockage of the filter, and the apparatus 2 can be configured to operate the fan 6 such that it can modulate its output to provide more performance if the pressure differential increase is detected thereby allowing the filter to operate efficiently to maintain air flow. Maintaining the air flow despite a clogged or blocked filter also advantageously maintains the heating and cooling capacity and therefore effectiveness of the present apparatus. Examples of fans that can be used in this embodiment include fans with brushless DC motors such as electronically commutated (EC) fans.

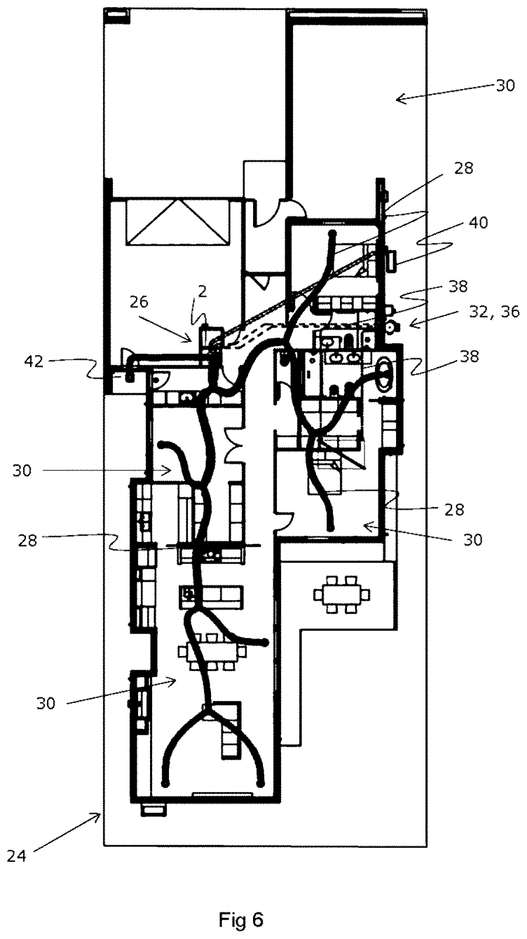

In the use of example systems illustrated in FIGS. 6 to 8, the system 24 for changing at least one characteristic of air and circulating that air has an apparatus 2 of FIGS. 1 to 5, where the apparatus 2 is located in a cavity 26 defined by the walls of the enclosed space 1. In a preferred embodiment, the apparatus 2 is accessible at wall level via an opening in a wall of the cavity 26. In a particularly preferred embodiment, the apparatus 2 is accessible at floor level. Alternatively, the apparatus 2 is mountable in a ceiling or floor space, arrangements which will be described in more detail in the following paragraphs.

Air from the outlet of the apparatus 4 is directed by a network of ducts 28 provided in a confined space, such as a roof space, to circulate the air into the enclosed space 1, for example, the different rooms 30 of a residential building, such as a kitchen, bedroom or living space, and then returned via the inlet 12 which is defined as multiplicity of apertures such as a mesh in the cover 16 of the FIGS. 1 and 2.

In a preferred embodiment, the apparatus 2 is arranged such that the cover 16 in the retracted or closed position occludes the opening in the wall thereby acting as a panel or fascia, and when the cover 16 is moved to the extended or open position, a user has access to the air characteristic changing means and/or air directing means 6, 8, 10 for facilitating servicing from within the enclosed space 1, such as a corridor as illustrated in FIG. 6. In particular, if the purification means 18 are located adjacent to the inlet 12 and on the inner surface of the cover 16, advantageously the purification means 18 can be easily cleaned, repaired or replaced from within a room 30 of the enclosed space 1. The cover 16 can be secured in the retracted position, for example by means of a fastener such as a latch or a soft close draw mechanism, although it can be appreciated that many types of fasteners could be used as known in the art.

Similarly, to the embodiment described in the previous paragraph, in an alternative embodiment, the apparatus 2 may be arranged so as to be mounted in the ceiling space, and where the cover 16 in the retracted or closed position occludes an opening in the ceiling surface, thereby also acting as a panel or fascia. The cover 16 can drop, under the influence of gravity, to an extended or open position, to allow the user to access the air characteristic changing means and/or air directing means for facilitating servicing of the apparatus 2. To prevent or reduce risk of injury, the apparatus can include a biasing means (not shown) for slowing the rate of movement of the cover into the open position or alternatively the biasing means can act to only allow movement of the cover when the user applies force to move it towards the closed or open position. In addition, the apparatus 2 may also have a locking means (not shown) for securing the cover 16 in the closed or retracted position for further reducing or preventing risk of injury.

In another alternative arrangement, the apparatus 2 may be mounted within a floor space and where the cover, when in the closed position, forms part of the floor and occludes an opening therein. When the cover is in the open position, the user can then access the apparatus for servicing, including repairing, replacement or maintenance of the filter(s), coils, fans or electronics.

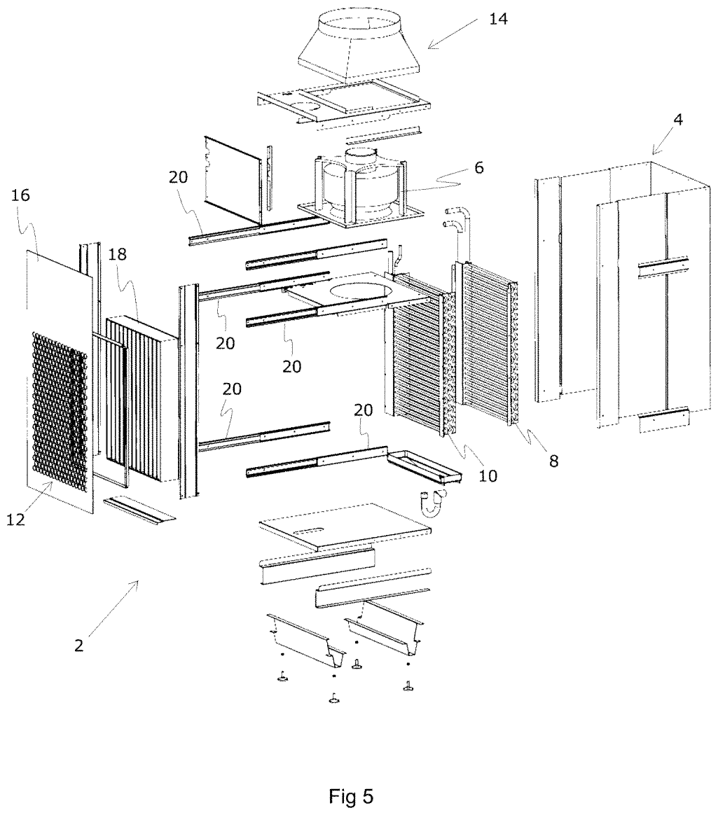

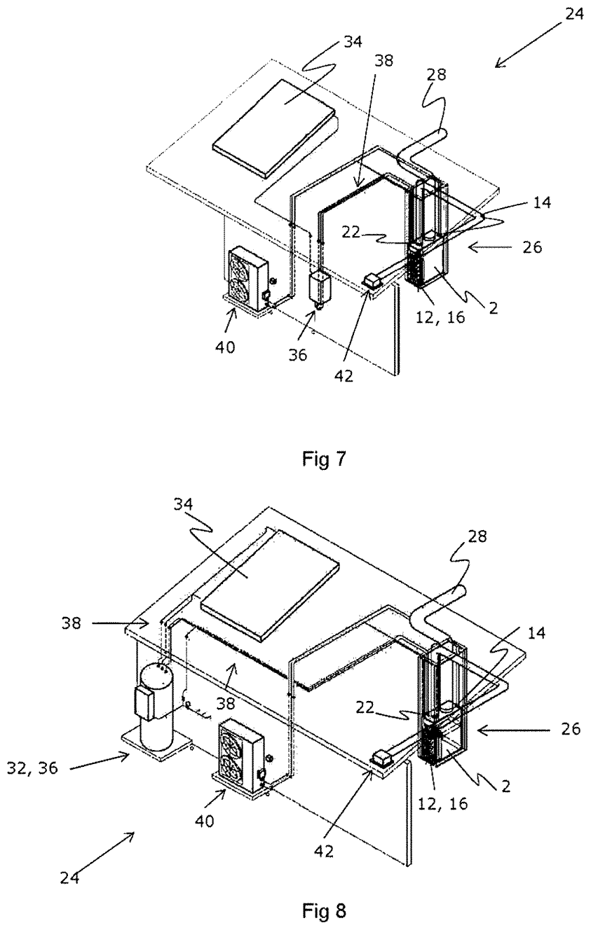

The air can be heated or cooled by passing it through the heating or cooling coils 8, 10 in the apparatus 2 which is directed through the outlet 14 and circulated within the enclosed space 1 via the ducts 28. Then the heating coil 8 can be coupled to heating systems 36, illustrated in FIG. 7 as a spontaneous hot water system and FIG. 8 as a gas hot water system. In particular the heating coil 8 can be coupled to a thermal bank 32, for example a water tank. In a preferred embodiment, the thermal bank 32 can store thermal energy harvested by solar panels 34 during the day and boosted by the heating system 36. The thermal bank 32 is connected to the apparatus 2 via water pipes 38. The cooling coil 10 can be coupled to a cooling system 40 exemplified in FIGS. 7 and 8 as an air conditioning condensing unit. While the example described in FIG. 5 describes separate heating and cooling coils 8, 10, in a preferred embodiment the apparatus may have a single refrigeration coil which can both heat and cool. However it can be appreciated that a variety of heating and cooling solutions could be coupled to heating and cooling coils 8, 10 to heat and cool air. Generally, the apparatus is also suitable to be coupled with a variety of energy sources, examples of such energy sources can include water or refrigeration energy sources.

The system 24 also has at least one sensor (not shown) which can detect different characteristics of air, examples of which include air temperature, pressure, humidity, pollutantlevels, CO, O₂, or CO₂ levels, air flow rate, hot water flow rate, refrigerant mass flow rate, heating or cooling coil temperature, and filter efficiency. The sensors can be located throughout the enclosed space 1, thereby providing information on a characteristic of air in a portion of the enclosed space 1 in proximity to the sensor(s). The at least one sensor can be located internal or external to the enclosure or within the apparatus. For example, the sensor(s) can be located external to the enclosure can detect the air temperature, pressure, humidity, pollutant levels or CO₂ levels, and at least one sensor internal to the enclosure can detect the air flow rate, hot water flow rate, refrigerant mass flow rate, heating or cooling coil temperature and air filter efficiency. In an example, at least one sensor can be provided in the ceiling space, floor space or external of the enclosed space, and to the building to determine the different air characteristics, such as the temperature of the external or ceiling space air, pollutant or CO₂ levels or humidity.

In an alternative embodiment, the system may be configured to receive signals from external to the system, i.e. signals which are associated with environmental monitoring, such as weather stations, air quality indexes, pollen count monitoring etc.

The sensors are coupled to a controller, which is in turn, coupled to the air characteristic changing means 8, 10, 18. Based on the signals received from the sensor(s) and/or inputs or external signals, for example a temperature set by a user on a thermostat, a controller can send signals to the one or more air characteristic changing means. The controller can comprise a processor and memory having executable instructions. For example, in response to a temperature sensor, the heating or cooling coil 8, 10 can be operated to change the temperature of the air or the air from the ceiling space or external of the enclosed space, and to the building can be selectably directed into the enclosure for optimum heat or cool recovery. In a preferred embodiment, in response to a pressure sensor which detects a pressure differential or resistance between opposing sides of the filter, the fan 6 can be operated to provide additional performance or an alert may be sent to a user for servicing or replacement. In such an embodiment, the fan is configured to be suitable for high resistance. Similarly, if CO₂ levels are high, outside air can be injected into the system 24 via an outside air intake 42 or the air flow rate can be increased. In this way, the system 24 operates to provide an environment in the enclosed space 1 which is managed to optimum levels of comfort and energy efficiency.

In particular, sensors can also detect if the purification means 18 is not working efficiently and at least one signal from the controller can be sent to a user to alert them to service or replace them. In a preferred embodiment, if it is detected that a new filter is required, the controller can send a signal to a filter supplier to place an order via an internet connection so that one or more filters can be delivered for convenient replacement. In another example, a sensor can be used to detect if the ultraviolet light source for producing ultraviolet light for the UVR or PCO purification means is not working and the controller can send a signal to a supplier to order a replacement for delivery via the internet connection. The sources for producing ultraviolet light can include lamps or ultraviolet light-emitting LEDs. As ultraviolet light-emitting LEDs have low operating costs, long service life, low maintenance and high efficiency, use of this technology is preferred in embodiments of the present invention.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments. In particular, while the embodiment illustrated in the Figures describes an apparatus that purifies the air and has components such as a fan and components for heating or cooling, it will be appreciated that the apparatus may be used only for purification of the air.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Complete listing of claims:

1. An apparatus for handling air in an enclosed space of a building and being mountable in structure of the building, the apparatus having an enclosure with a cover, an inlet formed in the cover and an outlet which is connectable to one or more ducts to return air to the enclosed space, the air being directable from the inlet to the outlet and from the outlet and, when the air is so directed, the apparatus is configured to change at least one characteristic of the air and wherein the cover is arranged to open to facilitate servicing from within the enclosed space wherein the arrangement for opening the cover is a slidable arrangement such that the cover and slidable arrangement thereby define a drawer for receiving purification means for purifying air on entry into the enclosure.

2. An apparatus according to claim 1, wherein the at least one characteristic of air includes one or more of the following: temperature, rate of air flow, humidity, air quality.

3. An apparatus according to claim 1, wherein the purification means is positioned internal to the cover and adjacent to the inlet.

4. An apparatus according to claim 1, being mountable in or at any one of the following: at floor level, in a cavity defined by walls of the enclosed space or in a roof or floor space.

5. An apparatus according to claim 1 wherein the apparatus is adapted to be accessible via an opening in the structure of the building wherein the slidable arrangement extends through the opening such that the cover is slidably movable from a retracted position where the cover occludes the opening and an extended position where the cover permits servicing of the apparatus.

6. An apparatus according to claim 5, wherein the structure of the building includes a wall(s), a ceiling or a floor.

7. An apparatus according to claim 5, wherein the cover is defined as a panel.

8. An apparatus according to claim 7, wherein the panel is a fascia having at least one opening.

9. An apparatus according to claim 5, wherein the cover is securable in the retracted position.

10. An apparatus according to claim 1, wherein changing the characteristic of air includes reduction of pollutant content for purifying the breathable air or changing the CO, CO₂or O₂ percentage content.

11. An apparatus according to claim 10, wherein the pollutant content includes particulates or volatile organic compounds and/or wherein the purification means utilises ultraviolet irradiation (UVR) and/or photocatalytic oxidation technology (PCO).

12. An apparatus according to claim 1, wherein the purification means is a filter, wherein the filter is one or more of a selection of: filter with a Minimum Efficiency Reporting Value (MERV) level of between 13 to 16, pre-filter, HEPA filter and carbon filter.

13. An apparatus according to claim 1, wherein the apparatus has a fan configured to be operable at high air resistances for directing air from the inlet to the outlet and any purification means so received therebetween, and wherein the apparatus is configured to detect a pressure differential or resistance across the received purification means, and when the pressure differential is at a pre-determined level, the apparatus is configured to operate the fan at a higher rate thereby maintaining the flow of air.

14. An apparatus according to claim 13, including a further inlet for allowing air external to the closed space or from the or a roof space to enter the enclosure and/or wherein the apparatus includes at least one purification means.

15. A system for handling air and circulating that air about an enclosed space of a building, the system having the apparatus according to claim 1, wherein a fan directs the air from the outlet via one or more ducts into the enclosed space into the inlet of the enclosure thereby circulating the air about the enclosed space.

16. A system according to claim 15, further including at least one sensor for detecting characteristics of the air and/or further including a controller coupled to the sensor(s) and air characteristic changing means, wherein the controller provides signals to the air characteristic changing means based on signals received from the sensor(s) or external signals associated with environmental monitoring to change the characteristic(s) of the air.

17. A system according to claim 16, wherein the sensor(s) is adapted to detect a reduction in efficiency of the purification means, and when the reduction is so detected, the sensor is adapted to provide a signal to the controller to send a further signal to service the purification means.

18.-20. (canceled)
